(19) United States
(12) Patent Application Publication
(10) Pub. No.: US 2008/0081989 A1
(43) Pub. Date: Apr. 3, 2008

(54) Medical examination or treatment facility

(75) Inventor: Bernd Maciejewski, Dormitz (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/903,150

(22) Filed: Sep. 20, 2007

(30) Foreign Application Priority Data
Sep. 29, 2006 (DE) 10 2006 046 318.8

(51) Int. Cl. A61B 5/05

(52) U.S. Cl. 600/410

(57) ABSTRACT

A medical examination or treatment facility, in particular a magnetic resonance facility, having a number of electronic modules and a number of casing elements encasing the facility on the outside, with at least some of the electronic modules and/or casing elements being fixed to support rails disposed on one or more components of the facility by means of quick-action tension elements that are guided in such a manner that they can be displaced longitudinally and can be secured on said rails.

### CROSS REFERENCE TO RELATED APPLICATIONS

This application claims priority of German application No. 10 2006 046 318.8 filed Sep. 29, 2006, which is incorporated by reference herein in its entirety.

### FIELD OF THE INVENTION

The invention relates to a medical examination or treatment facility, in particular a magnetic resonance facility, having a number of electronic modules and a number of casing elements encasing the facility on the outside.

### BACKGROUND OF THE INVENTION

Known examination or treatment facilities, in particular in the form of a magnetic resonance facility, but also for example in the form of a computed tomography unit, have a plurality of electronic modules in the form of larger, box-type module blocks, with a number of separate external casing elements being provided, to encase the facility on the outside. Both the electronic modules and the casing elements have to be detachable, the casing elements on the one hand so that the electronic modules can be reached for maintenance and repair purposes and the electronic modules on the other hand so that these can be removed and replaced as necessary. Generally the electronic modules and the casing elements are fixed to corresponding components of the facility, in the case of a magnetic resonance facility for example to corresponding supports on the magnet, by way of a plurality of separate screws. This type of fixing is extremely laborious for both assembly and disassembly purposes, since to remove one or more casing elements the fitter or service engineer has to loosen a plurality of screws manually, in order to reach the electronic modules located below, the removal of which is equally laborious. The same also applies in the case of assembly of course. It is not only laborious because a plurality of individual screws have to be tightened or loosened but it is also laborious because the electronic modules or casing elements have to be positioned very precisely to tighten the screws, so that the holes in the holding segments on the electronic modules or casing elements are positioned precisely above the threaded holes, into which the screws are to be screwed. The manner of fixing known to date is therefore very complex and very time-consuming. In the case of system maintenance in particular disassembly/assembly results in significant downtimes, in which the system is not available to the operator, so said operator is unable to use it profitably and the maintenance costs are of course very significant due to the extended length of time required for maintenance engineering.

### SUMMARY OF THE INVENTION

The invention is based on the problem of specifying an examination or treatment facility, in which at least some of the electronic modules and/or casing elements can be assembled or, as the case may be, disassembled in a simple manner.

To resolve this problem, provision is made with a medical examination or treatment facility of the type referred to in the introduction for at least some of the electronic modules and/or the casing elements to be fixed to support rails disposed on one or more components of the facility by means of quick-action tension elements that are guided in such a manner that they can be displaced longitudinally and can be secured on said rails.

The inventive fixing provided for the electronic modules/casing elements allows very fast and simple fixing, since only the quick-action tension elements have to be secured to the corresponding support rails for fixing purposes. Such quick-action tension elements, usually clamping elements, generally only require a very simple grip to pivot a clamp or similar, which can be achieved without significant effort. The quick-action tension elements are guided in such a manner that they can be displaced longitudinally on preferably horizontal support rails, in other words the quick-action tension elements and with them the electronic module/casing element, on which the quick-action tension elements are disposed, are displaced along the support rails and moved into the precise position, in which it is then secured by tensioning the quick-action tension elements.

In general the inventive connecting system of the quick-action tension elements disposed in a displaceable manner in the support rails and able to be clamped therein allows very simple assembly and disassembly of the electronic modules/casing elements and complex manual operations, as required to loosen the known screw connections, are not necessary here. It is also possible to fix the electronic modules/casing elements securely and in a fixed manner in the required position using only very few quick-action tension elements. Even large casing elements can be fixed without further ado to corresponding support rails by way of just four quick-action tension elements in the region of the corners, with no need for multiple screw joints along the longitudinal sides in the usual manner with the prior art to date due to the excellent holding action of the quick-action tension elements in conjunction with the support rails. The support rails can be of any length, although even very short support rails only a few centimeters in length and therefore only allowing very few screw joints are also conceivable.

In a particularly advantageous development of the invention the support rails are fixed to the component(s) with vibration damping by one or more intermediate damping elements. The support rails are therefore not screwed directly to the component(s), which would result in any vibration of the component, e.g. the magnet of a magnetic resonance facility, acting directly on the support rails and causing the electronic modules or casing elements to vibrate by way of these. Without damping the vibration is therefore transmitted directly by way of the fixing points to the casing for example and therefore to the patient chamber or the environment per se. Since according to the invention the support rails are supported on the component(s) by way of intermediate damping elements, each support rail can be decoupled completely in respect of vibration in a particularly advantageous manner. Component vibration is then only transmitted to the support rails with extremely effective damping, if at all, so that no significant vibration is transmitted to the electronic modules/casing elements.

The rails themselves are preferably embodied as C-profile rails with a clamping slot, into which clamping slot the quick-action tension element(s) engage(s) with a clamping action. A quick-action tension element itself preferably has a slot nut held in the clamping slot of the C-profile, which is pulled into the sides of the slot bounding the clamping slot during tensioning. To activate the clamping mechanism a quick-action tension element can have a pivoting clamping lever for example. Such a clamping lever is connected to a pin, to which the slot nut is in turn fixed. When the clamping lever, which has a corresponding rounded contact surface, is turned, the shape of the contact surface causes the pin and with it the slot nut to move axially, being thereby tensioned against the slot sides. Instead of a clamping lever, a quick-action tension element can also be configured as a jointed clamp with a lever and a jointed coupling to a clamping element held in the clamping slot, like the slot nut for example. A jointed clamp of this type in principle operates in a similar manner to a quick-action clamp with a clamping lever, but here the axial movement of the pin holding the slot nut is achieved by way of a joint connected to a toggle. Regardless of which type of quick-action tension element is used, it allows very simple operation with one hand, during which only one lever has to be turned to achieve firm tensioning, in order thereby to achieve an extremely firm clamping connection between the electronic module connected to the quick-action tension element and the support rail in a direct manner.

The inventive manner of fixing is particularly appropriate for larger or heavier electronic modules and relatively large casing elements, such as the front and rear casings or the side casings of the devices in question. According to an expedient development of the invention lighter or smaller electronic modules and casing elements can be fixed by way of plug-latch/rotary connection means comprising a first part disposed on a rail or a casing element and a second part disposed on the electronic module or casing element, which can be connected to the first part. These electronic modules and in particular smaller casing elements, such as for example the tower casing elements, which are disposed on the tower of a magnetic resonance facility, which allows the supply of cooling water, do not require such a firm fixing as heavy electronic modules and large casing elements, so simple plug-latch connection means, which can be closed and released in an equally quick and easy manner, are used here. For example the tower casing, which in the case of magnetic resonance devices has to be detachable, to reach the hitherto provided screw joints on the side or rear casing, which is partially covered by way of the tower casing, can be removed in this manner by simple pulling away, in other words unlatching, when using the inventive plug-latch connection means, which can be done much faster than the previous loosening of connecting screws. It is also possible here for example to provide the first part disposed on a support rail with an insertion hole, through which the second part disposed on the electronic module/casing element, in the form of a latching pin, is pushed and latched behind the hole for fixing purposes. The latching pin can be released again from this latch connection by simply pulling it in the counter direction or for example by a quarter turn using a tool or auxiliary tool. In principle however any plug-latch connection or plug-rotary connection can be used here, which allows simple fixing and simple release of an electronic module/casing element of this type.

It is also expedient to cover the quick-action tension elements of the casing elements fixed thereby by way of other casing elements, in some instances fixed by way of plug-latch-rotary connection means. In order for example to be able to fix a side casing element by way of the quick-action tension elements, these have to be disposed in a position, in which they can be operated manually by the fitter, for example by turning the clamping lever. They therefore have to be laterally offset to some degree in respect of the casing element on a corresponding support sheet of the casing element or similar. In order now to cover these quick-action tension elements for optical reasons, another casing element expediently overlaps the first-mentioned casing element in the region of the quick-action tension elements, so that these are no longer visible. It is of course particularly expedient for this second, covering casing element to be fixed by way of the similarly quick-release plug-latch connection means. The casing element here can be a casing element of the tower casing for example, which is pulled to some degree over the peripheral region of the side casing and therefore over the quick-action tension elements of the same. If the side casing should now be removed, the tower casing element simply has to be pulled away or the easy-fit elements have to be released, whereupon the quick-action tension elements of the side casing elements can be released immediately.

Finally it is also expedient to fix the quick-action tension elements of a number of electronic modules or casing elements to one rail. It is for example possible to fix both the side casing and the front or rear casing of a magnetic resonance facility for example to common rails, so that separate support rails do not have to be provided for this purpose.

### BRIEF DESCRIPTION OF THE DRAWINGS

Further advantages, features and details of the invention will emerge from the exemplary embodiments described below and with reference to the drawings, in which:

FIG. 1 shows a basic diagram of an inventive examination facility in the form of a magnetic resonance facility,

FIG. 2 shows a basic diagram of the fixing of a casing element to a support rail using a quick-action tension element, which is not yet secured,

FIG. 3 shows the diagram from FIG. 2 in the assembly position,

FIG. 4 shows a basic diagram of the fixing of different casing elements, and

FIG. 5 shows a basic diagram of a plug-latch-rotary connection means.

### DETAILED DESCRIPTION OF THE INVENTION

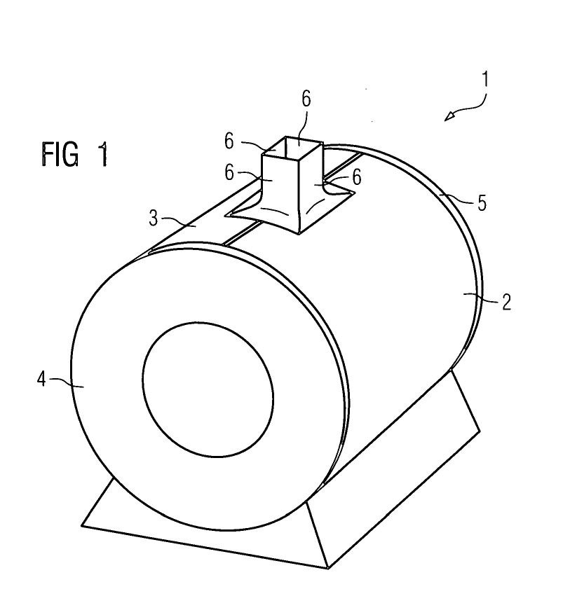

FIG. 1 shows an inventive examination facility 1 in the form of a magnetic resonance facility, which is structured in the manner known per se. Since the present invention only relates to the matter of fixing its electronic modules or casing elements in a specific manner, the magnetic resonance facility here is only shown in outline and there is no need to address the specific inner structure here. The inventive fixing of casing elements is described below with reference to the exemplary embodiments. It should be noted here that corresponding electronic modules, generally in the form of larger box-type assemblies, can also be fixed in the same manner.

FIG. 1 shows two side casings 2, 3, one front casing 4 and a rear casing 5, with the front and rear casings 4, 5 being represented as a single piece in the example shown. It is of course also possible to configure these as segmented in a number of pieces. Further casing elements, disposed tower-side and encasing the tower, which is used for cooling and is not shown in greater detail here, through which water and power are supplied, are also shown.

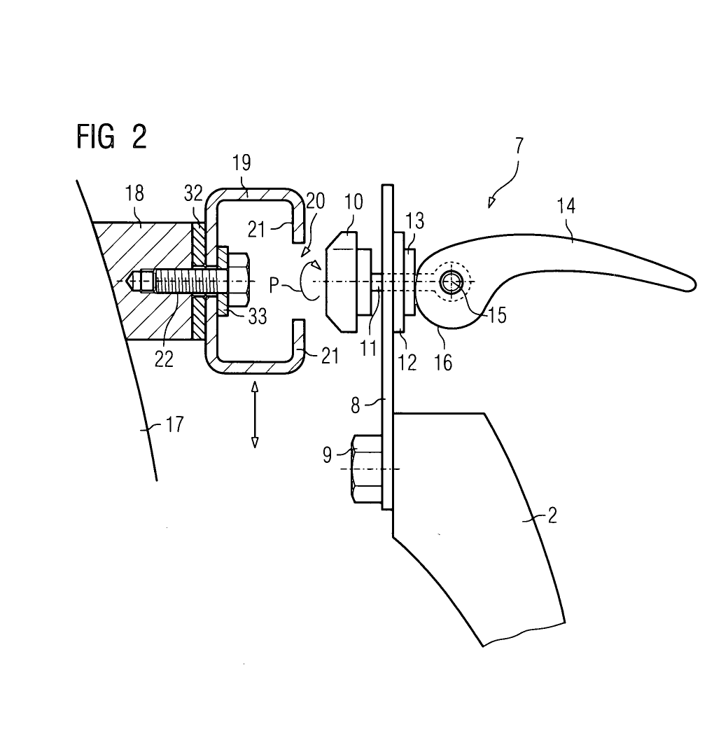

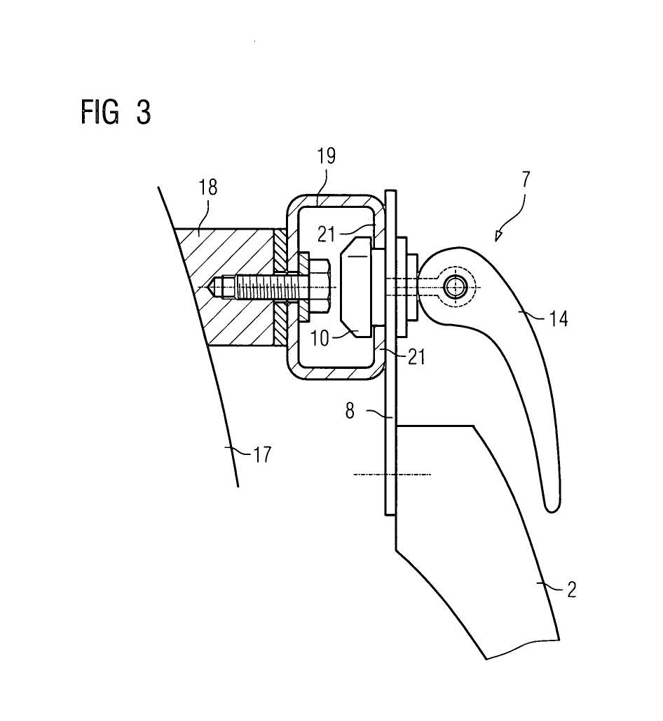

At least some of the described casing elements, primarily the side casings 2, 3 and the front and rear casings 4, 5, are fixed using quick-action tension elements. FIGS. 2 and 3 show basic diagrams illustrating the use of such a quick-action tension element 7. FIG. 2 shows the side casing element 2 by way of example, to which a fixing sheet 8 is fixed by way of a screw connection 9, so that it projects slightly to the side. The quick-action tension facility 7 is disposed on the fixing sheet 8. It comprises both a slot nut 10 or corresponding nuts, such as for example rhombus nuts, held in a clamping slot as described below. It can be rotated, as shown by the arrow P. To position the quick-action tension facility 7 in the support sheet 8, it is twisted a little, so that it can be passed through a sufficiently large hole in the support sheet 8. The slot nut 10 is disposed on a pin 11, which passes through the hole in the support sheet 8. Disposed on the opposite side of the support sheet 8 is a holding plate 12, for example a shim, followed by a further bearing element 13 for a clamping lever 14, which is shown here in the open position. The clamping lever 14 can now be pivoted about the axis 15 and is connected to the axis 15 by the pin 11. The clamping lever 14 has a curved edge 16 that rolls on the support element 13 in a manner known per se.

To fix the side casing element 2 a support rail 19 with a C-profile with a clamping slot 20, bounded by two profile sides 21, is disposed by way of a screw connection 22 on a component 17, in this instance the magnet for example, on a corresponding support 13. This support rail 19 preferably runs horizontally. The support rail is supported on the support 18 by way of an intermediate damping element 32, for example a foam rubber or plastic disk. The head of the connecting screw 22 is also supported by way of an intermediate damping element 33, again a rubber or plastic disk on the base of the support rail 19. This allows vibration decoupling to be achieved between the component 17 and the support rail 19 as well as the quick-action tension elements 7 fixed thereto.

To fix the side casing element 2 the slot nut 10 having an essentially rectangular form is first twisted a little, so that it can be inserted by its narrower side through the two slot sides 21 into the inside of the clamping slot 20. This twisting can be done for example by twisting the clamping lever 14, which is connected by way of the pin 11 to the slot nut 10, through 30°. The clamping lever 14 is then simply moved downward into the clamping position shown in FIG. 3. The asymmetrical form of the edge 16 means that the pin 11 and with it the slot nut 10 is drawn axially toward the support sheet 8. This causes the slot nut 10 to be pulled firmly against the slot sides 21. Therefore the side casing element 2 can be fixed to the support rail 19 by way of the quick-action clamping facility 7 using an extremely simple one-handed operation.

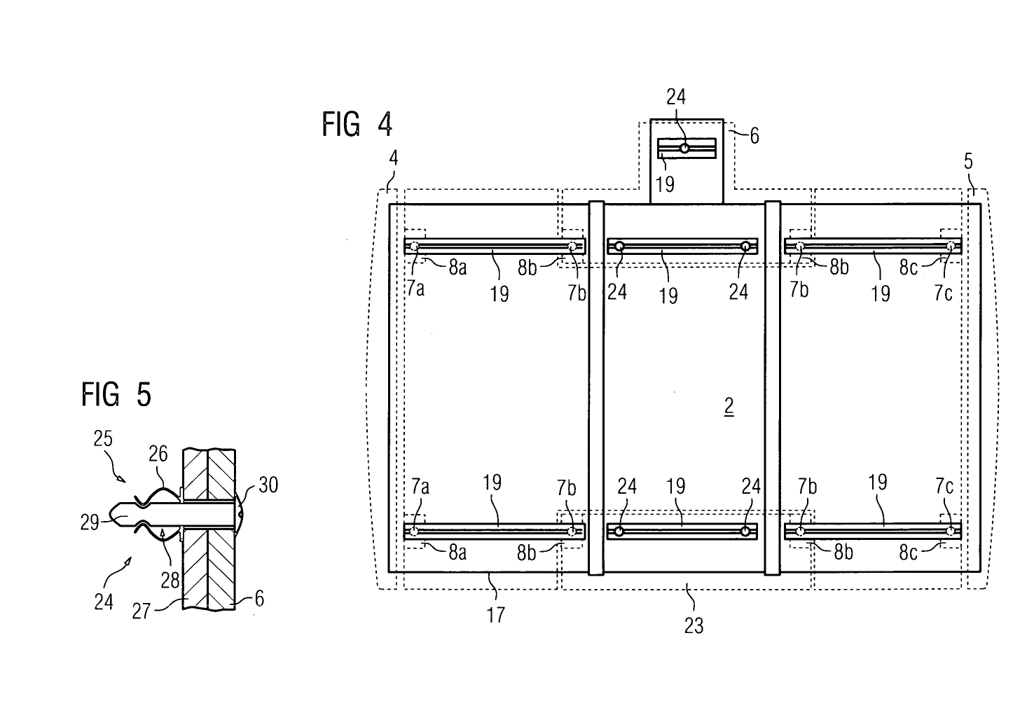

FIG. 4 shows a basic diagram of the magnet of a magnetic resonance facility as a component 17 holding different support rails 19. In the example shown six support rails 19 are provided. It shows the front casing 4, the side casing 2, the rear casing 5 and a tower casing 6 and a base-side casing 23 lying opposite this in the lower region of the magnet. The casing elements are only shown with broken lines here. The same applies to the support sheets 8 with the quick-action tension elements 7 located thereon, which due to the simpler assignment to the respective casing element are marked 7a, 7b, 7c and 8a, 8b, 8c. In the example shown the support sheets 8a and the quick-action tension elements 7a are located on the front casing, the support sheets 8b and the quick-action tension elements 7b are located on the side casing element and the support sheets 8c and quick-action tension elements 7c are located on the rear casing element 5.

For assembly purposes the front casing element 5 is positioned first for example and fixed to the two support rails 19 shown in the figure by way of the quick-action tension elements 7a. It is self-evident that corresponding support rails are of course also provided on the opposite side of the magnet, to which the front casing is fixed by way of corresponding quick-action tension elements 7a.

The rear casing 5 is then fixed, by tensioning the quick-action tension elements 7c in the support rails 19 adjacent thereto. In the next step the large side casing 2 is positioned and fixed by way of its quick-action tension elements 7b in the respective support rails 19. Before it is fixed the casing element can be displaced horizontally along the support rails 19. The side edges of the side casing 2 clearly overlap the support surface 8a and quick-action tension elements 7a as well as the support surface 8c and quick-action tension elements 7c of the front and rear casings 5, so these are no longer visible once the side casing 2 is in position. All that is still visible are the support sheets 8b and quick-action tension elements 7b belonging to the side casing 2. To cover these as well for optical reasons, in the next step the tower casing 6 and the lower casing element 23 are positioned, with the tower casing 6 for example being fixed on the one hand by way of plug-latch/rotary connection means 24 to the middle upper support rail 19 and to a further support rail 19 located on the tower. These plug-latch connection means are described in more detail below with reference to FIG. 5. The tower casing element 6 now visibly covers the two support sheets 8b and quick-action tension elements 7b, so that these are no longer visible either. The lower casing element 23 is fixed in a corresponding manner by way of plug-latch connection means 24 to a support rail 19 provided there. The support sheets 8b and quick-action tension elements 7b located there are therefore no longer visible.

Disassembly takes place in a correspondingly simple manner. In order to reach electronic modules or other assemblies (not shown in more detail here) located beneath the casing elements, only the tower casing element 6 (in some instance there may be more than one) and the lower casing element 23 (this is only optional) first have to be removed, which can be done by simply releasing the plug-latch connection means by pulling. Then just four quick-action tension elements 7b have to be released to remove the entire side casing element 2, after which the front or rear casing 4, 5 can also be removed as required. It should be noted here that the geometry of the casing elements shown is only of an exemplary nature to illustrate the operating principle. Naturally the geometry of the individual casing elements can be as required, as of course can the arrangement of the support sheets and quick-action tension elements. It is however important that it is possible in principle for the quick-action tension elements of different casing elements to be fixed to common support rails, as well as for a casing element to be able to cover the support sheets/quick-action tension elements of another casing element in the assembly position.

As described above, a plug-latch connection means 24 is used for the simplest possible assembly/disassembly of the relatively small, light casing elements 6 and (optionally) 23. Such a means is shown for example as a basic outline in FIG. 5. The plug-latch connection means comprises a first part 25, embodied here as an insertable element 26, for example in the form of an undercut leaf spring, and fixed here for example to a casing element 27, for example the side or rear casing. This insertable element 26, embodied for example as a leaf spring, is therefore quite elastic and has an undercut recess 28 for a latching head 29 of a latching pin 30, which in the example shown is fixed to the tower casing element 6 in a suitable manner or is passed through this. To fix the tower casing element 6 the respective latching pin 30 is now simply pushed into the hole 31 on the casing element 27, until the latching head 29 is disposed in the recess 28 and held by the shoulders bounding this. The latching pin 30 can be removed again from the elastic casing element 27 for disassembly purposes in a similarly simple manner, by means of a quarter turn using a tool as required.

1.-10. (canceled)

11. A medical device, comprising:
a support rail that disposed on a component of the device; and
a casing element that encases the device on an outside of the device and is fixed to the support rail by a quick-action tension element so that the casing element is displaced longitudinally and secured on the support rail.

12. The medical device as claimed in claim 11, wherein the support rail is disposed to run horizontally.

13. The medical device as claimed in claim 11, wherein the support rail is fixed to the component with a vibration damping by one or more intermediate damping elements.

14. The medical device as claimed in claim 11, wherein the support rail has a C-profile with a clamping slot where the quick-action tension element engages with a clamping action.

15. The medical device as claimed in claim 14, wherein a quick-action tension element has a slot nut held in the clamping slot of the C-profile that is pulled against slot sides bounding the clamping slot during tensioning.

16. The medical device as claimed in claim 14, wherein the quick-action tension element has a pivoting clamping lever.

17. The medical device as claimed in claim 14, wherein the quick-action tension element is a jointed clamp with a lever and a jointed coupling to a clamping element held in the clamping slot.

18. The medical device as claimed in claim 17, wherein the clamping element is held in the slot nut.

19. The medical device as claimed in claim 11, wherein the casing element is fixed to the support rail by a plug-latch/rotary connection.

20. The medical device as claimed in claim 19, wherein the plug-latch/rotary connection comprises a first part disposed on the support rail or the casing element and a second part disposed on the casing element connected to the first part.

21. The medical device as claimed in claim 11, wherein the quick-action tension element is covered by other casing elements fixed to the support rail by plug-latch connections.

22. The medical device as claimed in claim 11, wherein a plurality of quick-action tension elements of a plurality of casing elements are fixed to the support rail.

23. The medical device as claimed in claim 11, wherein the medical device comprises an electronic module that is fixed to the support rail by a further quick-action tension element.

24. The medical device as claimed in claim 11, wherein the medical device is a magnetic resonance device.

25. A method for assembling and disassembling a medical device, comprising:
disposing a support rail on a component of the device; and
fixing a casing element of the device to the support rail by a quick-action tension element for longitudinally displacing the casing element and securing the casing element on the support rail.

26. The method as claimed in claim 25, wherein the medical device comprises an electronic module that is fixed to the support rail by a further quick-action tension element.
